(19) United States
(12) Patent Application Publication
(10) Pub. No.: US 2008/0103305 A1
(43) Pub. Date: May 1, 2008

(54) Process for the preparation of imatinib

(76) Inventors: Peter MacDonald, Gentilino (CH); Pierluigi Rossetto, Balerna (CH)

Correspondence Address: KENYON & KENYON LLP, ONE BROADWAY, NEW YORK, NY, 10004, US

(21) Appl. No.: 11/978,227

(22) Filed: Oct. 26, 2007

Related U.S. Application Data
(60) Provisional application No. 60/854,774, filed on Oct. 26, 2006.
(60) Provisional application No. 60/874,420, filed on Dec. 11, 2006.
(60) Provisional application No. 60/958,367, filed on Jul. 5, 2007.
(60) Provisional application No. 60/963,238, filed on Aug. 2, 2007.
(60) Provisional application No. 60/967,617, filed on Sep. 5, 2007.
(60) Provisional application No. 60/995,332, filed on Sep. 25, 2007.
(60) Provisional application No. 60/860,624, filed on Nov. 22, 2006.
(60) Provisional application No. 60/979,256, filed on Oct. 11, 2007.
(60) Provisional application No. 60/934,911, filed on Jun. 14, 2007.
(60) Provisional application No. 60/997,849, filed on Oct. 5, 2007.

(51) Int. Cl. C07D 403/14

(52) U.S. Cl. 544/295

(57) ABSTRACT

The present invention provides process for the preparation of Imatinib and Imatinib salts, including processes that prepare intermediates for the production of Imatinib.

### CROSS REFERENCE TO RELATED APPLICATIONS

The present application claims the benefit of the following U.S. Provisional Patent Application Nos. 60/854,774, filed Oct. 26, 2006; 60/874,420, filed Dec. 11, 2006; 60/958,367, filed Jul. 5, 2007; 60/963,238, filed Aug. 2, 2007; 60/967,617, filed Sep. 5, 2007; 60/995,332, filed Sep. 25, 2007; 60/860,624, filed Nov. 22, 2006; 60/979,256, filed Oct. 11, 2007; 60/934,911, filed Jun. 14, 2007; and 60/TBA (Attorney Docket No. 13760/A403P2), filed Oct. 5, 2007. The contents of these applications are incorporated herein by reference.

### FIELD OF INVENTION

The present application relates to processes for the preparation of Imatinib, pharmaceutically acceptable salts thereof, and intermediates useful in the preparation of Imatinib.

### BACKGROUND OF THE INVENTION

Imatinib is an intermediate for the preparation of Imatinib salts, such as, Imatinib Mesylate. Imatinib Mesylate, 4-(4-methylpiperazin-1-ylmethyl)-N-[4-methyl-3-[(4-pyrinin-3-yl)pyrimidin-2-yloamino]phenyl]benzamide mesylate, a compound having the chemical structure,

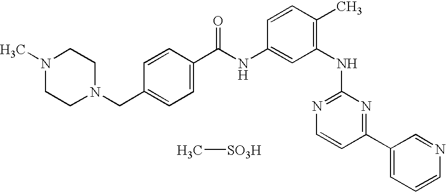

is a protein-tyrosine kinase inhibitor, especially useful in the treatment of various types of cancer and can also be used for the treatment of atherosclerosis, thrombosis, restenosis, or fibrosis. As such imatinib mesylate can also be used for the treatment of non-maligant diseases. Imatinib is usually administered orally in the form of a suitable salt, e.g., in the form of imatinib mesylate.

The preparation of Imatinib as reported in European Patent No. 0564409 describes a coupling reaction between N-(5-amino-2-methylphenyl)-4-(3-pyridyl)-2-pyridineamine and 4-[(4-methyl-1-piperazinyl)methyl]benzoyl chloride as illustrated by the following scheme:

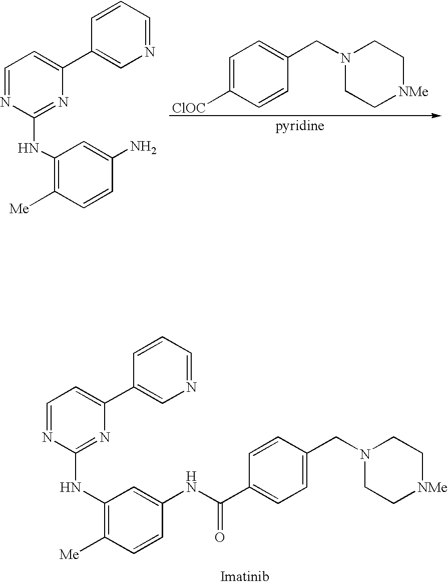

The above reaction is done in the presence of a high pyridine to starting amine (N-(5-amino-2-methylphenyl)-4-(3-pyridyl)-2-pyridineamine) ratio (about 138 equivalents which equals about 40 parts v/w), which leads to the use in such processes of a large quantity of pyridine, known to be a toxic solvent according to ICH guidelines. The work-up of the reaction is conducted by evaporation of the remaining pyridine, treatment with water and a slurring step in a dichloromethane/methanol mixture. The obtained product is then purified by chromatography, which is highly undesirable in processes on industrial scale because it is expensive and time consuming.

A similar synthetic approach is reported in more recently published patent applications, US patent application No. 2006/0149061 and US patent application No. 20060223817. These published applications describe the use of a similar pyridine/starting amine ratio (140 equivalents which equals about 41 parts v/w) and quantity of pyridine as described in European Patent No. 0564409. In addition, the processes described in the above publications also reports the recovery of the obtained product by evaporation of the remaining pyridine and subsequent extraction of the product from a basic aqueous phase with dichloromethane. The obtained product is then purified by slurrying in ethylacetate.

Another similar synthetic approach is reported in WO2004/074502. This publication describes the reaction of the amine (N-(5-amino-2-methylphenyl)-4-(3-pyridyl)-2-pyridineamine) with the acyl chloride (4-[(4-methyl-1-piperazinyl)methyl]benzoyl chloride) in an inert organic solvent, such as dimethylformamide (DMF), dimethylacetamide (DMA), N-methylpyrrolidone (NMP), sulfolane, diglyme, dioxane, and tetrahydrofuran (THF), providing the hydrohalide salt of imatinib, which is subsequently converted to Imatinib free base and then to Imatinib mesylate.

In above approaches a 4-[(4-methyl-1-piperazinyl)methyl]benzoyl chloride or a derivative thereof is used. In U.S. Pat. No. 4,623,486 (in preparation C) a process of preparing a salt of the 4-[(4-methyl-1-piperazinyl)methyl]benzoyl chloride is described. The above benzoyl chloride is prepared therein in EtOH, and the dihydrochloride is isolated. In addition, EP208404 (preparation. A) describes a process wherein the monohydrochloride thereof is isolated.

A different approach is described in US patent application No. 2004/0248918, and is illustrated by the following scheme:

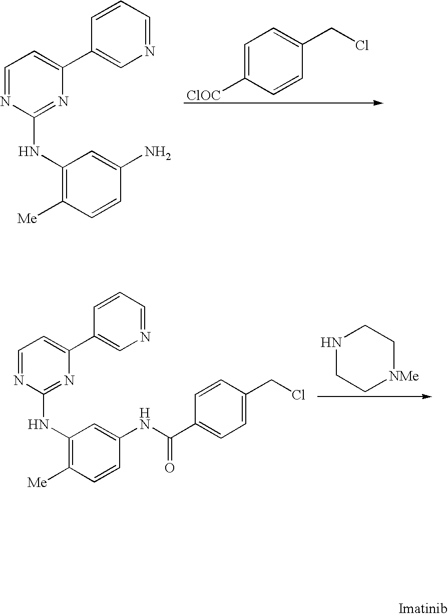

The last step of the reaction described in the above scheme is carried out in the presence of tetrahydrofuran (THF) as a reaction solvent and in the presence of pyridine as a base. The reaction is refluxed for 12 hours, and the product is purified by column chromatography (eluent: chloroform/methanol, 3:1 v/v), which is not a suitable purification method when performing the reaction on a large scale, followed by crystallization.

Thus, there exists a need for an alternative process for preparing Imatinib, that is suitable for scale-up, does not require the use of large quantities of pyridine and does not require the use of chromatography as a means of purification.

### SUMMARY OF INVENTION

In one embodiment, the present invention encompasses a process for preparing Imatinib of formula I

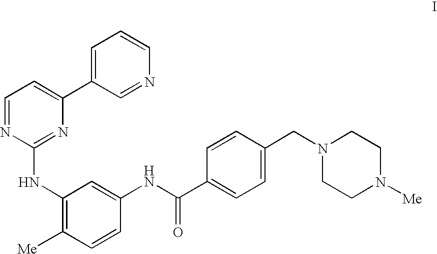

comprising:

a) reacting the amine of formula III, 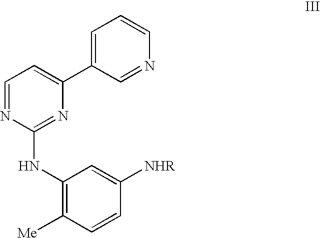
with a 4-[(4-methyl-1-piperazinyl)methyl]benzoyl derivative of formula IV 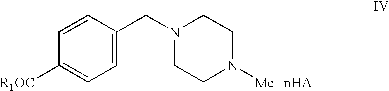
and an amount of about 2 to about 10 volumes (7 to 35 equivalents), preferably about 4 to about 7 volumes, more preferably about 5 to about 6 volumes per gram of the compound of formula III. of pyridine per gram of the compound of formula III; and

b) optionally recovering Imatinib of formula I;

wherein n is 0, 1, or 2; R₁ is a leaving group selected from the group consisting of: H, Cl, and Br, preferably R₁ is Cl; R is either H or a hydrocarbon group, preferably, H, and HA is an acid selected from the group consisting of: HCl, HBr, HI, Methanesulfonic acid, and para-toluenesulofinic acid, preferably HA is HCl.

In another embodiment, the present invention encompasses a process for preparing an Imatinib salt comprising preparing Imatinib of formula I by the process of the present invention, and converting it to an Imatinib salt. Preferably, the Imatinib salt is Imatinib mesylate.

In yet another embodiment, the present invention encompasses a process for preparing 4-[(4-methyl-1-piperazinyl)methyl]benzoic acid of formula II, comprising:

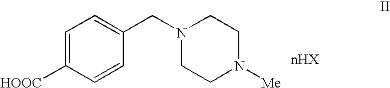

a) reacting a 4-benzoic acid derivative of the following formula 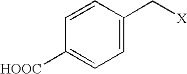
with N-methylpiperazine of the following formula (preferably about 4-5 equivalents), and

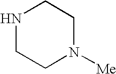

b) optionally recovering the 4-[(4-methyl-1-piperazinyl)methyl]benzoic acid of formula II; wherein X is a leaving group selected from the group consisting of Cl, Br, I, mesyloxy and tosyloxy, preferably X is Cl; n is 0, HX is an acid selected form the group consisting of: HCl, HBr, HI, Methanesulfonic acid, para-toluenesulfonic acid, preferably HA is HCl.

In another embodiment, the present invention encompasses a process for preparing Imatinib salt of the following formula

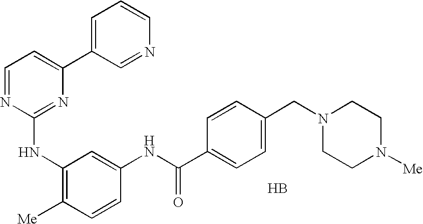

comprising preparing the 4-[(4-methyl-1-piperazinyl)methyl]benzoic acid of formula II by the process of the present invention, and converting it to Imatinib salt; wherein HB is an acid, preferably, methanesulfonic acid.

### DETAILED DESCRIPTION OF INVENTION

The present invention is related to processes for preparing Imatinib, intermediates thereof, and pharmaceutical acceptable salts thereof. These processes of the present invention provide Imatinib in high yields and purity. Also, these processes can be adapted easily to industrial scale because, when using pyridine as a solvent, it is present in small amounts, and the recovery of a substantially pure product is simple and not time consuming.

The processes can be illustrated by the following scheme: 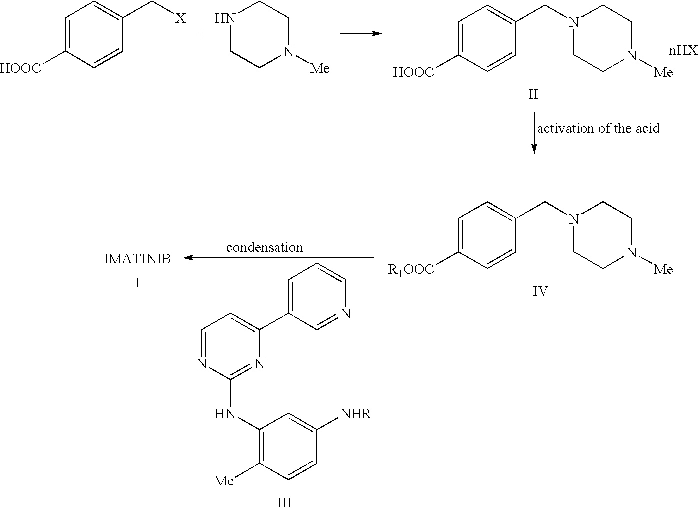
wherein X is Cl, Br, I, mesyloxy or tosyloxy, preferably X is Cl; n is 0, 1 or 2, preferably n=0; HX is an acid selected form the group consisting of: HCl, HBr, HI, Methanesulfonic acid, and para-toluenesulofinic acid, preferably HX is HCl; R₁ is a leaving group selected from the group consisting of: H, Cl, and Br; and R is either H or a hydrocarbon group, preferably, H.

Preferably, the hydrocarbon group is an alkyl or aryl group. Preferably, the alkyl group is optionally, substituted by a hetero atom. More preferably, the alkyl group is a C₃₋₈ cyclo-alkyl, a C₄₋₈ cyclo alkenyl, or a C₃₋₈ alkoxy. Preferably, the aryl group is phenyl.

The first step in these processes comprises preparing a 4-[(4-methyl-1-piperazinyl)methyl]benzoic acid of formula II. 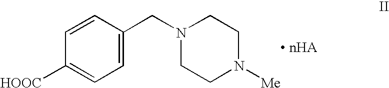
This process comprises

a) reacting a 4-benzoic acid derivative of the following formula 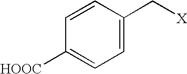
with N-methylpiperazine of the following formula,

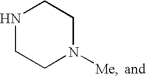

b) optionally recovering 4-[(4-methyl-1-piperazinyl)methyl]benzoic acid of formula II; wherein X is a leaving group selected from the group consisting of Cl, Br, I, mesyl or tosyl, preferably X is Cl, n is 0, and HX is an acid selected form the group consisting of: HCl, HBr, HI, Methanesulfonic acid, and para-toluenesulofinic acid, preferably HX is HCl.

The amount of N-methylpiperazine in the reaction of step a) is about 3 to about 6, preferably about 4 to about 5 equivalents of the amount of the benzoic acid derivative with which it is reacted.

In the above process of the present invention, the reaction is done in the presence of an organic solvent. Preferably, the organic solvent is a protic organic solvent, more preferably, an alcohol, even more preferably, a C₁₋₆ alcohol, more preferably, methanol, ethanol, n-propanol, iso-propanol, n-butanol, iso-butanol, sec-butanol, n-pentanol, iso-pentanol, sec-pentanol, n-hexanol, and mixtures thereof, most preferably, n-butanol.

Combining the two reactants and the solvent provides a solution. The solution is maintained at a temperature of about 15° C. to about 30° C., preferably of about 20° C. to about 25° C. Preferably, the solution is maintained for about 2 to about 10 hours, more preferably for about 3 to about 6 hours; during this time 4-[(4-methyl-1-piperazinyl)methyl]benzoic acid of formula II is expected to be formed.

The compound of formula II may be recovered by any known process, preferably by evaporating the solvent from the above mixture; adding a protic organic solvent to obtain a second mixture; heating the second mixture at a temperature of about 70° C. to about 90° C., preferably of about 70° C. to about 82° C., more preferably, to a temperature of about 80° C. to about 82° C.; cooling the heated second mixture to obtain a precipitate, and filtering the precipitate.

Preferably, the organic solvent is a protic organic solvent, more preferably, an alcohol, even more preferably, a C₁₋₆ alcohol, most preferably, methanol, ethanol, n-propanol, iso-propanol, n-butanol, iso-butanol, sec-butanol, n-pentanol, iso-pentanol, sec-pentanol, n-hexanol, and mixtures thereof, and even most preferably, iso-propanol.

Preferably, the heated second mixture is cooled to a temperature of about 15° C. to about 30° C., more preferably of about 20° C. to about 25° C., to obtain a precipitate. The recovery may further comprise washing the filtered precipitate, and drying.

The process for preparing 4-[(4-methyl-1-piperazinyl)methyl]benzoic acid of formula II may further comprise the conversion of 4-[(4-methyl-1-piperazinyl)methyl]benzoic acid of formula II to an Imatinib salt of the following formula; 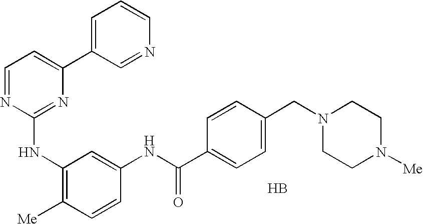
wherein HB is an acid, preferably, methanesulfonic acid. The use of the compound of formula II instead of its acid salt form improves the performance of the process for preparing Imatinib or salt thereof due to its solubility in the reaction medium.

The conversion of the compound of formula II to imatinib salt can be carried out for example, by the process disclosed in European Patent 208404, preparation P. This process includes a step where a hydrochloride salt of the acid of formula II is converted to the activated acid derivative 4-[(4-methyl-1-piperazinyl)methyl]benzoyl derivative of formula IV or salt thereof of the following formula, 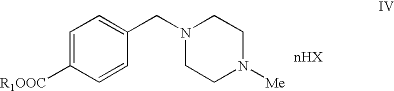
where X and R₁ are described before and the compound of formula is isolated.

In a preferred embodiment, the reaction for preparing imatinib from the 4-[(4-methyl-1-piperazinyl)methyl]benzoyl derivative of formula IV or salt thereof comprises 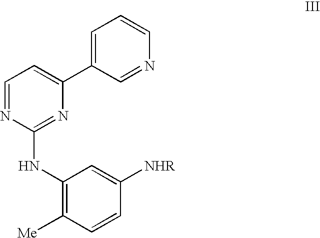
with a 4-[(4-methyl-1-piperazinyl)methyl]benzoyl derivative of formula IV or salt thereof 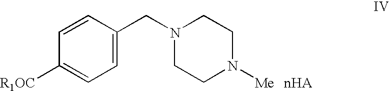
and about 2 to about 10 volumes (7 to 35 equivalents) preferably about 4 to about 7 volumes, more preferably about 5 to about 6 volumes per gram of pyridine per gram of the compound of formula III; and

b) optionally recovering Imatinib of formula I;

wherein n is 0, 1, or 2; R₁ is a leaving group selected from the group consisting of: H, Cl, Br, mesyl and tosyl, preferably, R₁ is Cl; R is either H or a hydrocarbon group, preferably, H, and HA is an acid selected form the group consisting of: HCl, HBr, HI, Methanesulfonic acid, para-toluenesulofinic acid, preferably, the acid is HCl.

The reaction is done in the presence of a minimum amount of pyridine, which is about 2 to about 10 volumes (7 to 35 equivalents) preferably about 4 to about 7 volumes, more preferably about 5 to about 6 volumes per gram, which may serve as a solvent and as a base.

The amine of formula III is combined with pyridine to obtain a solution. To this solution a 4-[(4-methyl-1-piperazinyl)methyl]benzoyl derivative of formula IV is then added. This addition may be done at low temperatures to avoid the formation of impurities. Preferably, the addition is done at a temperature of about 0° C. to about 25° C., more preferably of about 15° C. to about 25° C.

The addition provides a reaction mixture. Preferably, the reaction mixture is maintained at a temperature of about 10° C. to about 30° C., more preferably of about 15° C. to about 25° C. Preferably, the reaction mixture is maintained for about 30 minutes to about 4 hours, more preferably for about 1 hour; during this time the formation of Imatinib salt of having the following formula, 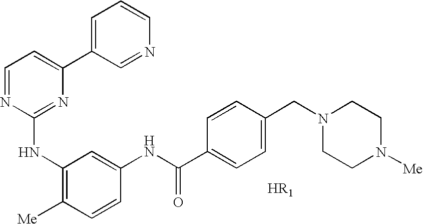
occurs; wherein R₁ is derived from the compound of formula IV, preferably, Cl. Imatinib is recovered from the said mixture by a process comprising: admixing water with the reaction mixture comprising the Imatinib salt, and reacting with a base.

Preferably, an aqueous solution of the base is used. Preferably, the base is selected from the group consisting of ammonium hydroxide, sodium hydroxide, and potassium hydroxide, preferably ammonium. Preferably, before the addition of the base heating to a temperature of about 30° C. to about 50° C., more preferably of about 40° C., is conducted. Heating may be carried out to obtain a solution. The addition of the base provides Imatinib, which precipitates by the addition of an additional amount of water. Preferably, after adding the second amount of water, the mixture is maintained at 15° C. to about 25° C., to increase the yield of the precipitated Imatinib. In addition, to increase the yield even more, the mixture is maintained for an overnight period, preferably the overnight period is about 12 hours to about 16 hours

The recovery process of Imatinib may further comprise filtering off the precipitated Imatinib, washing and drying.

The starting material, 4-[(4-methyl-1-piperazinyl)methyl]benzoyl derivative, can be the free base when n is 0, or the corresponding salt derivative when n is either 1 or 2. Accordingly, when n when n is 2, and X is Cl, the compound of formula IV corresponds 4-[(4-methyl-1-piperazinyl)methyl]benzoyl dihydrochloride of the following formula.

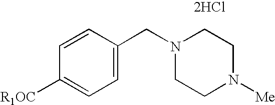

R₁ in the compound of formula IV is a leaving group as defined above, preferably R₁ is Cl. Accordingly, when n is 0 and R₁ is Cl, the compound of formula IV corresponds to 4-[(4-methyl-1-piperazinyl)methyl]benzoyl chloride of the following formula.

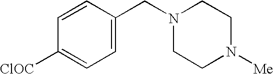

When n is 2, and R₁ is Cl, the compound of formula IV corresponds to 4-[(4-methyl-1-piperazinyl)methyl]benzoyl chloride dihydrochloride of the following formula.

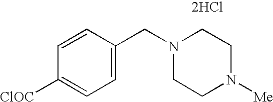

The free base, 4-[(4-methyl-1-piperazinyl)methyl]benzoyl derivative of formula IV, may be obtained according to the process described before in the present application or by any process known to one skilled in the art. The salt is, usually, a hydrochloride salt, preferably, dihydrochloride. The dihydrochloride salt can be obtained from a commercial source.

The process for preparing Imatinib can further comprise the conversion of Imatinib to imatinib salt. Preferably, the salt is a mesylate salt. The conversion of Imatinib to Imatinib salt can be done by reacting Imatinib with an acid, as exemplified in U.S. application Ser. No. 11/796,573, filed Apr. 27, 2007.

The conversion can be carried out for example by combining imatinib base with a mixture of a C₁-C₄ alcohol, preferably ethanol, and water. The temperature can be lowered to below room temperature, such as about −10° C.-0° C. A source of MeSO₃H, such as a solution of MeSO₃H in a C₁-C₄ alcohol is then added. The reaction mixture can be seeded. The reaction mixture can then be maintained to increase the yield of the mesylate. The mesylate can be recovered by evaporating solvents from the reaction mixture to obtain a residue.

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

### Example 1

### Preparation of Imatinib

To a solution of N-(5-amino-2-methylphenyl)-4-(3-pyridyl)-2-pyridineamine (80 g) in pyridine (400 g) at 0° C., 4-[(4-methyl-1-piperazinyl)methyl]benzoyl chloride dihydrochloride (1.1 eq) is added. The reaction is kept under stirring at 15-20° C. for 1 h, then water (400 mL) is added. The mixture is heated up to 40° C., then 26% NH₄OH (200 g) and water (900 g) are added. The reaction mixture is kept under stirring at room temperature overnight. The solid is filtered off, washed with water and dried at 75° C. under vacuum for 3-4 h. Imatinib is obtained as a yellowish powder (135 g, 95% yield, >98% purity).

### Example 2

### Preparation of Imatinib

To a suspension of 4-[(4-methyl-1-piperazinyl)methyl]benzoic acid (84 g) in pyridine (400 g) at 0° C., SOCl₂ (44.8 g, 1.05 eq) is added and the mixture is kept under stirring at 30-50° C. for 1-2 h. After cooling at 0° C., N-(5-amino-2-methylphenyl)-4-(3-pyridyl)-2-pyridineamine (80 g) is added. The reaction is kept under stirring at 15-20° C. for 1 h, then water (400 mL) is added. The mixture is heated up to 40° C., then 26% NH₄OH (200 g) and water (900 mL) are added. The reaction mixture is kept under stirring at room temperature overnight. The solid is filtered off, washed with water and dried at 75° C. under vacuum overnight. Imatinib is obtained as a yellowish powder (125 g, 88% yield, >98% purity).

### Example 3

### Preparation of Imatinib

To a suspension of 4-[(4-methyl-1-piperazinyl)methyl]benzoic acid dihydrochloride (30 g) in pyridine (100 g) at 20° C., SOCl₂ (11.5 g, 1.05 eq) is added and the mixture is kept under stirring at 45-50° C. for 1-2 h. After cooling at 0° C., N-(5-amino-2-methylphenyl)-4-(3-pyridyl)-2-pyridineamine (20 g) is added. The reaction is kept under stirring at 15-25° C. for 1 h, then water (100 mL) is added. The mixture is heated up to 40° C., then 26% NH₄OH (50 g) and water (225 mL) are added. The reaction mixture is kept under stirring at room temperature overnight. The solid is filtered off, washed with water and dried at 75° C. under vacuum overnight. Imatinib is obtained as a yellowish powder (32 g, 90% yield, <98% purity).

### Example 4

### Preparation of Imatinib

To a suspension of 4-[(4-methyl-1-piperazinyl)methyl]benzoic acid (10 g) in CH₂Cl₂ (400 g) at room temperature, DCC (9.6 g) and HOBT (9 g) are added. After 18 h stirring, the solid is filtered off and washed with CH₂Cl₂ (100 g). N-(5-amino-2-methylphenyl)-4-(3-pyridyl)-2-pyridineamine (9.5 g) is added to the combined filtrates, the solution is stirred at 15-25° C. for 1 h, then DMAP (1 g) is added and stirring is continued for 2 days. After addition of water (200 g) and 26% NH₄OH (20 g), the organic phase is separated and evaporated. The residue is taken up with IPA (100 g). The product is filtered, washed with EPA and dried (13.5 g, 77% yield, 96.3% purity).

### Example 5

### (Synthesis of 4-[(4-methyl-1-piperazinyl)methyl]benzoic acid

4-(Chloromethyl)benzoic acid (58 g) is added to a solution of N-methylpiperazine (150 g) in n-BuOH (580 g) at room temperature. After stirring for 3-6 h, the solvent is evaporated under reduced pressure and the residue is taken up with IPA (440 g). The mixture is refluxed for 15 min under stirring, then stirred for 24 h at room temperature. The solid is filtered off, washed with IPA (2×58 g) and dried under vacuum at 70° C. overnight. The desired product is obtained as a white solid (59.5 g, 75% yield).

### Example 6

### Synthesis of Imatinib mesylate According to U.S. Pat. No. 6,894,051

4-[(4-Methyl-1-piperazinyl)methyl]-N-[4-methyl-3-[[4-(3-pyridinyl)-2-pyrimidinyl]aminophenyl]benzamide (98.2 g) is added to EtOH (1.4 L). To the suspension methanesulfonic acid (19.2 g) is added dropwise. The solution is filtered clear at 65° C. The solvent is evaporated and the residue is taken up with EtOH (2.2 L) and dissolved under reflux with addition of water (30 mL). The solution is cooled down and kept overnight at 25° C. The solid is filtered off and dried at 65° C. The title product is obtained as light beige crystals.

### Example 7

### Synthesis of 4-[(4-methyl-1-piperazinyl)methyl]benzoyl chloride dihydrochloride

To a suspension of compound II (n=2, A=Cl) (20 g) in toluene (35 mL) and DMF (1 mL) under N2 at 60° C., (20 g) was added over a period of 1 h SOCl2. The mixture was kept under stirring at 62° C. for 20 h. After cooling at 20° C., toluene (20 mL) was added and the mixture was stirred for 0.5 h. The solid was filtered off, washed with toluene (50 mL) and dried at 65° C. under vacuum for 15 h. The product was obtained as a white powder (21 g).

### Example 8

### Preparation of Imatinib Mesylate

Imatinib base (60 g, 0.1216 mol) was suspended in EtOH (900-1200 mL) and water (2-5% v/v vs EtOH) was added under stirring. The temperature was adjusted to −10/−5° C. and a solution of MeSO₃H in EtOH (79.8 mL 10% v/v; 0.1213 mol) was added in 2 min, keeping the temperature at −10/−5° C.

The reaction mixture was seeded with Imatinib mesylate form X (300-500 mg) and kept under stirring at −5° C. for 3 h. The suspension was diluted with MTBE (750-1000 mL) keeping the temperature below 0° C. The solid was filtered off, washed with MTBE and dried under vacuum onto the filter in a nitrogen atmosphere to remove free EtOH. Crystalline Imatinib mesylate containing about 7% EtOH was obtained in 92-95% yield.

### Example 9

### Preparation of Imatinib Mesylate

Imatinib base (60 g; 0.1216 mole) was suspended in 1200 ml of Ethanol and stirred. Reactor was kept under flow of nitrogen during all of the experiment (6 litres per hour). Then, 24 ml of water was added to the suspension and the temperature was adjusted at −15° C. An ethanolic solution of methanesulfonic acid (79.8 ml 10% V/V; 0.1213 mole) was added during 2 minutes to the reaction mixture. Temperature of the solution was set at −10° C. during 10 minutes, imatinib base was dissolved and seeding material of form X (2 g) was added. The crystallization process was continued under stirring for 190 minutes and temperature was continuously increased to −5° C. The suspension was stored overnight in a freezer at approx. −27° C. Than, suspension was diluted by 1000 ml TBME, filtered by nitrogen pressure and obtained crystalline portion was washed with 400 ml TBME. The resulted crystalline form was dried by flow of nitrogen through the filter to remove free ethanol. Ethanol content was about 7.5%. (Yield was 67.95 g; 85%)

What is claimed is:

1. A process for preparing Imatinib of formula I comprising: 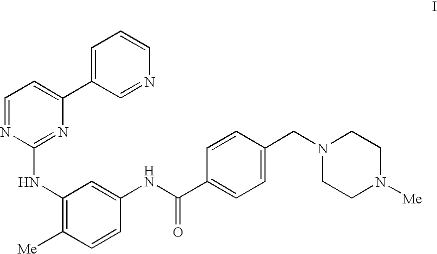
reacting the amine of formula III, 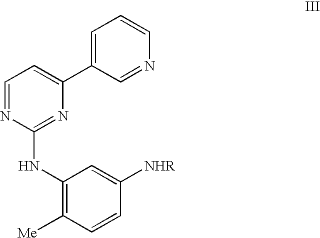
with a 4-[(4-methyl-1-piperazinyl)methyl]benzoyl derivative of formula IV 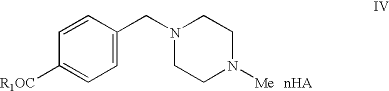
and pyridine in an amount of about 2 to about 10 volumes per gram of the compound of formula III;
wherein n is 0, 1, or 2; R₁ is a leaving group selected from the group consisting of: H, Cl, Br, mesyl and tosyl; R is either H or a hydrocarbon group; and HA is an acid.

2. The process of claim 1, wherein R₁ is Cl and n=0.

3. The process of claim 1, wherein R₁ is Cl and n=2.

4. The process of claim 1, wherein the 4-[(4-methyl-1-piperazinyl)methyl]benzoyl derivative of formula IV is prepared by
a) reacting a 4-benzoic acid derivative of the following formula 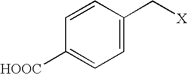
with N-methylpiperazine of the following formula, and 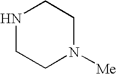
to obtain the 4-[(4-methyl-1-piperazinyl)methyl]benzoic acid of formula II 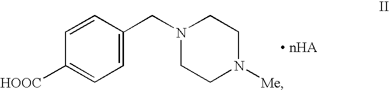
wherein n=0; and
b) converting the 4-[(4-methyl-1-piperazinyl)methyl]benzoic acid of formula II to obtain the 4-[(4-methyl-1-piperazinyl)methyl]benzoyl derivative of formula IV.

5. The process of claim 1, wherein a 4-[(4-methyl-1-piperazinyl)methyl]benzoyl derivative of formula IV or a salt thereof is added to a solution of the amine of formula III in pyridine at a temperature of about 0° C. to about 25° C. to obtain a reaction mixture.

6. The process of claim 5, wherein the reaction mixture is maintained at a temperature of about 10° C. to about 30° C. to obtain imatinib salt of the following formula

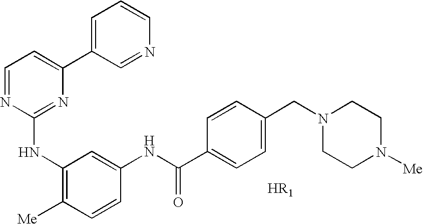

7. The process of claim 1, further comprising the step of recovering Imatinib from a product mixture comprising a salt of Imatinib having the following formula, 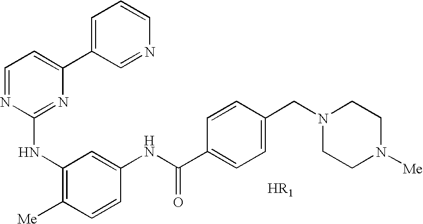
wherein R₁ is derived from the compound of formula II comprising the steps of:
admixing water with the product mixture, and reacting the imatinib salt with a base to obtain imatinib.

8. The process of claim 7, wherein the base is an inorganic base.

9. The process of claim 8, wherein the inorganic base is selected from the group consisting of ammonium hydroxide, sodium hydroxide, and potassium hydroxide.

10. The process of claim 9, wherein the inorganic base is ammonium.

11. The process of claim 7, wherein Imatinib is precipitated by the addition of an additional amount of water.

12. The process of claim 1, further preparing an Imatinib salt comprising converting Imatinib of formula I to an Imatinib salt.

13. The process of claim 9, wherein the salt is a mesylate salt.

14. The process of claim 1, wherein the pyridine is about 4 to about 7 volumes per gram of the compound of formula III.

15. The process of claim 1, wherein the pyridine is about 5 to about 6 volumes per gram of the compound of formula III.

16. A process for preparing 4-[(4-methyl-1-piperazinyl)methyl]benzoic acid of formula II, comprising: 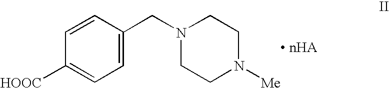
reacting a 4-benzoic acid derivative of the following formula 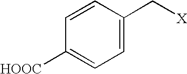
with N-methylpiperazine of the following formula, and 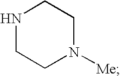
wherein X is Cl, Br, I, mesyl or tosyl, and n is 0.

17. The process of claim 16, wherein X is Cl and n=0.

18. The process of claim 16, wherein the reaction in step a) is carried out in a protic organic solvent.

19. The process of claim 18, wherein the protic organic solvent is a C₁₋₆ alcohol.

20. The process of claim 19, wherein the C₁₋₆ alcohol is selected from the group consisting of, methanol, ethanol, n-propanol, iso-propanol, n-butanol, iso-butanol, sec-butanol, n-pentanol, iso-pentanol, sec-pentanol, n-hexanol, and mixtures thereof, most preferably, n-butanol.

21. The process of claim 20, wherein the C₁₋₆ alcohol is n-butanol.

22. The process of claim 18, wherein the solution of the two reactants and the protic organic solvent is maintained at a temperature of about 15° C. to about 30° C. to obtain the compound of formula II.

23. The process of claim 22, wherein the temperature is about 20° C. to about 25° C.

24. The process of claim 16, wherein further comprising recovering the 4-[(4-methyl-1-piperazinyl)methyl]benzoic acid of formula II.

25. The process of claim 24, wherein the recovering step b) comprises
a) evaporating the solvent from the above mixture;
b) adding a protic organic solvent to obtain a second mixture;
c) heating the second mixture at a temperature of about 70° C. to about 90° C.;
d) cooling the heated second mixture to obtain a precipitate; and
e) filtering the precipitate to obtain the 4-[(4-methyl-1-piperazinyl)methyl]benzoic acid of formula II.

26. The process of claim 24, further comprising preparing Imatinib salt of the following formula 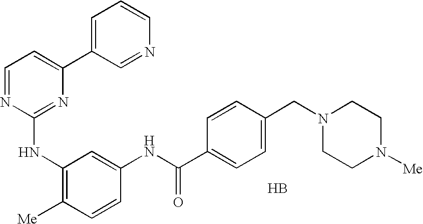
by converting the 4-[(4-methyl-1-piperazinyl)methyl]benzoic acid and salts thereof of formula II to an Imatinib salt; wherein HB is an acid.

27. The process of claim 26, wherein HB is methanesulfonic acid.
